(19) United States
(12) Patent Application Publication
(10) Pub. No.: US 2021/0369693 A1
(43) Pub. Date: Dec. 2, 2021

(54) METHODS AND PHARMACEUTICAL COMPOSITIONS FOR THE TREATMENT OF NON-ALCOHOLIC STEATOHEPATITIS

(71) Applicant: Eiger Biopharmaceuticals, Inc., Palo Alto, CA (US)

(72) Inventor: David CORY, Palo Alto, CA (US)

(21) Appl. No.: 16/768,000

(22) PCT Filed: Nov. 27, 2018
(86) PCT No.: PCT/US2018/062645

§ 371 Date: May 28, 2020

Related U.S. Application Data
(60) Provisional application No. 62/591,667, filed on Nov. 28, 2017.

(51) Int. Cl. A61K 31/4418; A61K 31/198; A61P 1/16; A61K 9/00

(52) U.S. Cl. CPC A61K 31/4418; A61K 9/0053; A61P 1/16; A61K 31/198

(57) ABSTRACT

In one aspect, methods of treating non-alcoholic steatohepatitis (NASH) or preventing or delaying the progression of non-alcoholic fatty liver disease (NAFLD) to NASH are provided. In some embodiments, the method comprises administering a therapeutically effective amount of pirfenidone. In some embodiments, the method comprises administering therapeutically effective amounts of pirfenidone and ubenimex as part of a combination therapy.

### FIELD OF THE INVENTION

The present disclosure provides methods and compositions for the treatment of non-alcoholic steatohepatitis (NASH) and prevention of progression to fibrosis and cirrhosis of the liver and hepatocellular carcinoma (HCC) resulting therefrom, and so relates to the fields of medicine, medicinal chemistry, pharmacology, chemistry, and biology.

### BACKGROUND OF THE INVENTION

Fatty liver, also known as fatty liver disease (FLD) or hepatic steatosis, is a reversible condition in which large vacuoles of triglyceride fat accumulate in liver cells via the process of steatosis (i.e., abnormal retention of lipids within a cell). Despite having multiple causes, fatty liver is most commonly associated with excessive alcohol consumption and obesity. FLD associated with other diseases that influence fat metabolism is referred to as “non-alcoholic” FLD or “NAFLD.”

Fatty change represents the intracytoplasmatic accumulation of triglycerides (neutral fats). At the onset of FLD, the hepatocytes present small fat vacuoles (liposomes) around the nucleus (microvesicular fatty change). In late stages of FLD, the size of the vacuoles increase and vacuoles coalesce to produce irreversible fatty cysts or lesions. Liver disease with extensive inflammation and a high degree of steatosis often progresses to more severe forms of the disease.

Non-alcoholic steatohepatitis (NASH) is an extreme and progressive form of NAFLD that is not linked to alcohol consumption and is further accompanied by inflammation (hepatitis). NASH is accompanied by ballooning degeneration of hepatocytes (also referred to herein as “hepatocyte ballooning”), which refers to the increase in size (i.e., ballooning) of cells during this process that is considered to be a form of apoptosis. Ballooned cells are typically two to three times the size of adjacent hepatocytes and characterized by a wispy cleared cytoplasm on H&E stained sections. Liver cell death and the inflammatory response lead to activation of stellate cells, which play a pivotal role in hepatic fibrosis. Further disease progression leads to cirrhosis and hepatocellular carcinoma (HCC), resulting in liver failure and, ultimately, death.

For patients suffering from early stages of NASH, lifestyle intervention, such as significant weight reduction, may slow or even reverse the process of steatosis. However, for patients with advanced NASH, there are no currently available therapies. For example, a recent clinical study of cenicriviroc for the treatment of advanced NASH failed to meet its primary endpoint of improving inflammation and liver damage after a year of treatment. Given the severity of FLD and NASH and unmet clinical need, an effective therapeutic treatment is urgently needed.

### BRIEF SUMMARY OF THE INVENTION

In one aspect, the present disclosure provides methods, materials, and pharmaceutical compositions, for treating and preventing the progression of FLD and NASH. It is envisaged that the methods and compositions described herein can slow or prevent NAFLD patients from progressing to NASH and can be used to treat NASH patients with beneficial effects of slowing, stopping, or reversing NASH disease progression in those patients.

In one aspect, the present disclosure relates to pharmaceutical compositions and methods for delivery of pirfenidone (5-methyl-1-phenyl-2-1-(H)-pyridone; also known as 5-methyl-1-phenyl-2-(1H)-pyridone, or Esbriet) as marketed by Genentech, Inc.) to treat FLD and NASH patients and to prevent and/or slow the progression of the disease to fibrosis, cirrhosis, HCC, and death. In many embodiments, these compositions for oral administration are formulated as immediate release preparations, e.g. tablets, capsules, or pills, and are conveniently packaged, for example, in the form of the pill bottles or blister packaging, for patients or their care providers to administer in therapeutically effective amounts.

In some embodiments, pirfenidone is administered in the treatment of NASH in accordance with the disclosure by oral administration of doses ranging from 300 mg per day up to 4005 mg per day at dosing frequencies of once (QD administration), twice (BID administration), or thrice per day (TID administration). In some embodiments, administration of doses of from about 267 mg to about 1200 mg administered from once to thrice daily is efficacious in the treatment of the majority of FLD and NASH patients. In some embodiments, a recommended dose of 801 mg is administered thrice daily. In one embodiment, a recommended dose of 2403 mg is administered once daily in an extended release formulation.

In some embodiments, a patient receives continuous daily dosing of pirfenidone, with a patient taking a therapeutically effective dose at least once per day for an extended period of time. In some embodiments, a new patient receives titrated dosages of pirfenidone over a 14-day period. For example, a patient may receive a first dosage of 267 mg TID (801 mg/day) for treatment days 1 through 7, a second dosage of 534 mg TID (1602 mg/day) for treatment days 8-14, and an ongoing dosage of 801 mg TID (2403 mg/day) for treatment days 15 and onward. In some embodiments, a patient receives a maximum daily dose of up to 3600 mg. In some embodiments, a patient receives 399 mg TID (1197 mg/day).

Measureable symptomatic improvement or a detectable slowing of disease progression may not occur until after several weeks or more of treatment. Clinical trials to demonstrate efficacy will likely run for at least 52 and more likely 72 or more weeks of treatment. Accordingly, patients may be treated for multiple consecutive days, weeks (e.g., at least 2 weeks), months, or years, including for the rest of the subject's life.

In some embodiments, a patient is administered a leukotriene A4 hydrolase (LTA4H) inhibitor comprising ubenimex in a combination therapy for at least some portion of the time they receive pirfenidone therapy. In some embodiments, such treatments in accordance with the disclosure can reduce hepatocyte ballooning and/or inflammation in the patient to generate biochemical and histological improvements in NASH.

In some embodiments, methods for treating NASH are provided, said methods comprising administration of pirfenidone to a patient in need of treatment. In some embodiments, the pirfenidone is administered orally. In some embodiments, the pirfenidone is administered in liposome formulation by parenteral administration. In some embodiments, the pirfenidone is administered at a daily dosage of 3600 mg, 2403 mg, or less. In some embodiments, the daily dosage is from about 801 mg to approximately 2403 mg. In some embodiments, the daily dosage is in the range of about 267 mg to about 2403 mg and is administered QD, BID, or TID, wherein the daily dosage is administered using unit dosage forms comprising pirfenidone in an amount of 267 mg, 399 mg, or 801 mg. In some embodiments, the daily dosage is administered BID and each dose is administered at a dose of approximately 130 mg, 140 mg, 150 mg, 160 mg, 170 mg, 180 mg, 190 mg, 200 mg, 250 mg, 300 mg, 400 mg, 450 mg, 500 mg, 550 mg, 600 mg, 650 mg, 700 mg, 750 mg, 798 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg. In some embodiments, the daily dosage is administered TID and each dose is administered at a dose of 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 250 mg, 300 mg, 399 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, or 1500 mg. In some embodiments, the pirfenidone is administered for at least 15 days, for at least 1 month, for at least 16 weeks, for at least 26 weeks, for at least 52 weeks, for at least 72 weeks, for at least a period of time greater than 72 weeks, or for the remainder of the subject's life. In some embodiments, treatment reduces fibrosis in the patient. In some embodiments, treatment reduces hepatocyte ballooning in the patient. In some embodiments, treatment reduces inflammation in the patient.

In some embodiments, methods for reducing hepatocyte ballooning in a NAFLD or NASH patient are provided, said method comprising administering pirfenidone at a daily dosage of from 801 to 3600 mg using consecutive daily dosing for at least 15 days. In some embodiments, the pirfenidone is administered for at least 52 weeks.

In some embodiments, methods for decreasing inflammation and/or fibrosis in a NASH patient are provided, said method comprising administering pirfenidone at a daily dosage of from 801 to 3600 mg using consecutive daily dosing for at least 72 weeks. In some embodiments, the pirfenidone is administered for the remainder of the subject's life.

In some embodiments, methods for the treatment or prevention of non-alcoholic steatohepatitis or its progression are provided, said method comprising administration of pirfenidone and optionally a secondary pharmaceutical agent comprising ubenimex to a patient in need thereof. In some embodiments, the pirfenidone is administered at a daily dosage of 4005 mg or less and the ubenimex is administered at a daily dosage of 450 mg or less. In some embodiments, the pirfenidone is administered at a dosage of 801 mg TID and the ubenimex is administered at a dosage of 150 mg TID. In some embodiments, the pirfenidone and the ubenimex are administered for at least 2 weeks, and preferably at least 52 weeks, at least 72 weeks, or the remainder of the subject's life.

In another aspect, the disclosure also provides for the manufacture of a medicament for the treatment of NASH and/or for prevention of the progression of NAFLD to NASH, wherein the active ingredient in the medicament is pirfenidone. In some embodiments, the medicament is a pharmaceutical composition comprising pirfenidone and at least one pharmaceutically acceptable carrier. In various embodiments, the medicament is formulated for oral administration, including immediate release and sustained release pharmaceutical formulations. The disclosure also provides for the manufacture of unit dosage forms of the medicament useful in treating patients and pharmaceutical packs and kits comprising one or more containers with a solid or liquid formulation of pirfenidone as described herein.

In some embodiments, pharmaceutical preparations comprising pirfenidone for the treatment of non-alcoholic steatohepatitis are provided. In some embodiments, the pharmaceutical preparation is in the form of a tablet, capsule, or pill suitable for oral administration. In some embodiments, the pharmaceutical preparation comprises pirfenidone in an amount ranging from about 267 mg to about 1200 mg. In some embodiments, the pharmaceutical preparation is in the form of a liposome suitable for parenteral administration. In some embodiments, the pharmaceutical preparation comprises pirfenidone in a concentration ranging from about 50 mg/ml to about 3600 mg/ml.

In another aspect, methods of treating non-alcoholic steatohepatitis (NASH) are provided, comprising administering to a subject in need of treatment a therapeutically effective amount of pirfenidone. In some embodiments, the subject has early-stage or middle-stage NASH. In some embodiments, the pirfenidone is administered at a total daily dose in the range of 100 mg to 5000 mg. In some embodiments, the pirfenidone is administered at a daily dose of about 801 mg to about 2403 mg. In some embodiments, the pirfenidone is administered at a daily dose of about 100 mg to about 5000 mg QD. In some embodiments, the pirfenidone is administered at a daily dose of about 50 mg to about 2500 mg BID. In some embodiments, the pirfenidone is administered at a daily dose of about 400 mg to about 1200 mg BID. In some embodiments, the pirfenidone is administered for at least 15 days. In some embodiments, the pirfenidone is administered for at least 52 weeks. In some embodiments, treatment results in a reduction in plasma CK-18 levels in the subject. In some embodiments, treatment results in a reduction in hepatocyte ballooning in the subject. In some embodiments, the method of treating NASH comprises administering pirfenidone in combination with ubenimex.

In another aspect, methods of delaying or preventing the progression of NAFLD to NASH in a subject having NAFLD are provided. In some embodiments, the method comprises administering to the subject a therapeutically effective amount of pirfenidone. In some embodiments, treatment results in a reduction in hepatocyte ballooning in the subject. In some embodiments, the pirfenidone is administered at a total daily dose in the range of 100 mg to 5000 mg. In some embodiments, the pirfenidone is administered at a daily dose of about 801 mg to about 3600 mg. In some embodiments, the pirfenidone is administered at a daily dose of about 100 mg to about 5000 mg QD. In some embodiments, the pirfenidone is administered at a daily dose of about 50 mg to about 2500 mg BID. In some embodiments, the pirfenidone is administered at a daily dose of about 400 mg to about 1200 mg BID. In some embodiments, the pirfenidone is administered for at least 15 days. In some embodiments, the pirfenidone is administered for at least 52 weeks. In some embodiments, the method of delaying or preventing the progression of NAFLD to NASH comprises administering pirfenidone in combination with ubenimex.

In still another aspect, methods of decreasing hepatocyte ballooning in a subject having NAFLD and/or NASH are provided. In some embodiments, the method comprises administering to the subject a therapeutically effective amount of pirfenidone for at least 15 days. In some embodiments, the method comprises administering to the subject a therapeutically effective amount of pirfenidone for at least 52 weeks. In some embodiments, the method comprises administering to the subject a therapeutically effective amount of pirfenidone for the remainder to the subject's life. In some embodiments, the subject has NAFLD. In some embodiments, the subject has NASH.

In yet another aspect, methods of decreasing inflammation in a subject having NAFLD and/or NASH are provided. In some embodiments, the method comprises administering to the subject a therapeutically effective amount of pirfenidone for at least 15 days. In some embodiments, the method comprises administering to the subject a therapeutically effective amount of pirfenidone for at least 52 weeks. In some embodiments, the method comprises administering to the subject a therapeutically effective amount of pirfenidone for the remainder of the subject's life. In some embodiments, the subject has NAFLD. In some embodiments, the subject has NASH.

In yet another aspect, methods of decreasing fibrosis in a subject having NASH are provided. In some embodiments, the method comprises administering to the subject a therapeutically effective amount of pirfenidone for at least 15 days. In some embodiments, the method comprises administering to the subject a therapeutically effective amount of pirfenidone for at least 52 weeks. In some embodiments, the method comprises administering to the subject a therapeutically effective amount of pirfenidone for the remainder of the subject's life.

In yet another aspect, pharmaceutical packages comprising unit dosage forms of pirfenidone and further comprising unit dosage forms of ubenimex are provided. In some embodiments, each unit dosage form of pirfenidone comprises pirfenidone in an amount from 100 mg to 5000 mg, and each unit dosage form of ubenimex comprises ubenimex in an amount from about 30 mg to 450 mg. In some embodiments, each unit dosage form of pirfenidone comprises pirfenidone in an amount from about 267 mg to about 801 mg. In some embodiments, each unit dosage form of ubenimex comprises ubenimex in an amount from about 75 mg to about 150 mg. In some embodiments, the pirfenidone and ubenimex are each formulated for immediate release. In some embodiments, the pirfenidone and ubenimex are each formulated for controlled release. In some embodiments, the pirfenidone and ubenimex are in the form of a tablet, a capsule, or a pill. In some embodiments, at least one of the pirfenidone and the ubenimex is in the form of a liposome.

### DETAILED DESCRIPTION OF THE INVENTION

### II. INTRODUCTION

Nonalcoholic fatty liver disease (NAFLD) is a spectrum of liver disease that ranges from simple steatosis to non-alcoholic steatohepatitis (NASH), fibrosis, and cirrhosis. The key histological features of NASH include steatosis, hepatocyte ballooning, and lobular inflammation, and fibrosis is also typically observed. Takahashi et al., World J Gastroenterol, 2014, 20:15539-15548.

As described in the Examples section below, it is hypothesized that in an animal model of NASH, pirfenidone demonstrates efficacy in slowing and preventing hepatocyte ballooning and reduces steatosis and lobular inflammation. Accordingly, in one aspect, methods of treating one or more symptoms of NAFLD and/or NASH, such as hepatocyte ballooning, steatosis, and lobular inflammation, are provided. Furthermore, Example 1 hypothesizes that plasma CK-18 levels decline significantly relative to those measured in control animals. Thus, in some embodiments, administration of pirfenidone as described herein is hypothesized to decrease ballooning and measurably lower plasma and/or liver CK-18 levels in as few as 3 to 12 weeks after treatment initiation. In some embodiments, it is hypothesized that continued daily administration of pirfenidone is efficacious in decreasing inflammation and fibrosis in NASH within 2 to 72 weeks after treatment initiation. In some embodiments, it is hypothesized that treatment with pirfenidone results in an improvement in one or more parameters such as improved ALT enzyme levels, decreased inflammation, decreased steatosis, reduced severity of NASH symptoms, reduced levels of NASH biomarkers such as CK-18, or the slowing, stopping, or reversing of liver fibrosis.

### II. DEFINITIONS

The terminology used herein is for the purpose of describing particular embodiments only and is not intended to be limiting. In this specification and in the claims that follow, reference will be made to a number of terms, which shall be defined to have the definitions set forth below. Unless otherwise defined, all technical and scientific terms used herein have the same meaning as commonly understood by one of ordinary skill in the art to which this invention belongs. The singular forms also include the plural unless the context clearly dictates otherwise. Thus, the singular forms “a,” “an,” and “the” include plural referents unless the context clearly dictates otherwise.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

Acronyms. The following acronyms are used throughout the specification and defined as follows: NASH: non-alcoholic steatohepatitis; NAFLD: non-alcoholic fatty liver disease; QD: one a day; BID: twice a day; TID: three times a day.

As used herein, “active agent” refers to a compound or drug that exerts a preventative or therapeutic effect on a disease or condition. As used herein, “active agent” can refer to either a single active agent or to a combination of two or more different active agents.

The terms “administer” and “administration” refer to a method of delivering a compound, a composition, or an agent to the desired site of biological action. These methods include, but are not limited to, oral delivery, intravenous delivery, parenteral delivery, intramuscular delivery, intraperitoneal delivery, or subcutaneous delivery.

As used herein, “capsules” are unit dosage forms (e.g., for oral administration) in which the active agent-containing composition may be encapsulated in the form of a liquid or solid (including particulates such as granules, beads, powders or pellets). Suitable capsules may be either hard or soft, and are typically made of gelatin, starch, or a cellulosic material. In some embodiments, the capsules are gelatin capsules.

The term “compound” refers to a molecule and encompasses not only the specified molecular entity but, if the compound is an active agent or drug, also its pharmaceutically acceptable, pharmacologically active analogs, including, but not limited to, active metabolites, amides, conjugates, esters, hydrates, polymorphs, prodrugs, salts, solvates, and other such derivatives, analogs, including deuterated analogs and analogs containing radioactive atoms or other labeling moieties, and related compounds.

The term “comprising” is intended to mean that the compounds, compositions and methods include the recited elements, but not excluding others. “Consisting essentially of,” when used to define compounds, compositions and methods, means excluding other elements that would materially affect the basic and novel characteristics of the claimed invention. “Consisting of” means excluding any element, step, or ingredient not specified in the claim. Embodiments defined by each of these transition terms are within the scope of this invention.

The term “CK-18” refers to cytokeratin-18 fragment, which has been identified as a noninvasive biomarker for NASH in that it is markedly increased in patients with NASH as determined by histology and higher blood plasma levels of the fragment correlate with the odds of having fibrosis on liver biopsy. See, Feldstein et al., Hepatology, 2009, 50:1072-8, incorporated by reference herein.

The term “dosage form” refers to a form of a pharmaceutical composition for administration to a subject (e.g., a human or non-human animal having a disease or condition to be treated). “Dose” refers to an amount of active agent. “Unit dosage form” refers to a dosage form that contains a fixed amount of active agent. For example, a single tablet or capsule is a unit dosage form. In some embodiments, multiple unit dosage forms are administered to provide a therapeutically effective dose.

The term “oral unit dosage form,” as used herein, refers to a unit dosage form that is intended to be orally administered.

The terms “effective amount” and “therapeutically effective amount” refer to an amount of an active agent being administered that will treat to some extent a disease, disorder, or condition, e.g., relieve one or more of the symptoms of the disease being treated (e.g., NASH), and/or that amount that will prevent, to some extent, one or more of the symptoms of the disease that the subject being treated has or is at risk of developing. For example, for a given parameter, a therapeutically effective amount will show an increase or decrease of therapeutic effect of at least 5%, 10%, 15%, 20%, 25%, 30%, 40%, 50%, 60%, 70%, 80%, 90%, or 100%. Therapeutic efficacy can also be expressed as “-fold” increase or decrease. For example, a therapeutically effective amount can have at least a 1.2-fold, 1.5-fold, 2-fold, 5-fold, or more effect over a control.

An “excipient” or “carrier,” as used herein, refers to a biologically inactive substance used in combination with an active agent(s) of the formulation. An excipient can be used, for example, as a solubilizing agent, a stabilizing agent, a diluent, an inert carrier, a preservative, a binder, a disintegrant, a coating agent, a flavoring agent, or a coloring agent. A wide variety of pharmaceutically acceptable excipients, such as vehicles, adjuvants, carriers or diluents, and auxiliary substances, such as pH adjusting and buffering agents, tonicity adjusting agents, stabilizers, wetting agents and the like, are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The term “liposome,” as used herein, refers to a spherical vesicle having at least one lipid bilayer and comprising one or more pharmaceutical agents in accordance with the present invention.

The term “parenteral administration,” as used herein, refers to a non-oral means of administration, and includes subcutaneous, intravenous, intramuscular, and inhalation routes of administration.

The term “pharmaceutical composition” refers to a composition that is suitable for administration to a subject. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral and/or parenteral.

The term “pharmaceutically acceptable,” as used with reference to a compound or component, means that the compound or component is generally safe, non-toxic, and not biologically undesirable. When the term “pharmaceutically acceptable” is used herein to refer to a pharmaceutical carrier or excipient, it is implied that the carrier or excipient has met the required standards of toxicological and manufacturing testing or that it is included on the Inactive Ingredient Guide prepared by the U.S. Food and Drug Administration.

The term “pharmaceutically acceptable salt” refers to a derivative of an active agent produced by making acid or base salts thereof. Examples of pharmaceutically acceptable salts include, but are not limited to, mineral or organic acid salts of basic residues such as amines, alkali or organic salts of acidic residues such as carboxylic acids, and the like. Pharmaceutically acceptable salts include the conventional non-toxic salts or the quaternary ammonium salts of the parent compound formed, for example, from non-toxic inorganic or organic acids. Pharmaceutically acceptable salts include those formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Pharmaceutically acceptable salts include solvent addition forms (solvates) or crystal forms (polymorphs) as defined herein, of the same salt.

The terms “prevention,” “preventing,” and “prevent” mean avoiding the onset of a clinically evident disease progression altogether or slowing the onset of a pre-clinically evident stage of a disease in individuals at risk. Prevention includes prophylactic treatment of those at risk of developing a disease.

The term “sign,” as used herein, means an indication of disease and includes conditions that can be observed by a doctor, nurse, or other health care professional.

The terms “subject” and “patient” interchangeably refer to a human or non-human animal (e.g., mammal) suitable for treatment with an active agent. A subject in need thereof may have a disease (e.g., NASH) or may be at an increased risk, relative to the general population, of developing a disease (e.g., NASH). In some embodiments, a subject has been diagnosed with a disease (e.g., NASH).

The term “symptom” means a sign or other indication of disease, illness, or injury. Symptoms may be felt or noticed by the individual experiencing them or by others, including by non-health-care professionals.

The terms “treatment,” “treating,” and “treat” refer to any indicia of success in the treatment or amelioration of an injury, disease, or condition, including any objective or subjective parameter such as abatement, remission, improvement in patient survival, increase in survival time or rate, diminishing of symptoms or making the injury, disease, or condition more tolerable to the patient, slowing in the rate of degeneration or decline, or improving a subject's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

“Pirfenidone” refers to 5-methyl-1-phenyl-2-1-(H)-pyridone; also known as 5-methyl-1-phenyl-2-(1H)-pyridone, the structure of which is shown below:

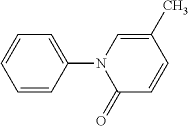

Pirfenidone belongs to the chemical class of pyridine. Pirfenidone has a molecular formula of C₁₂H₁₁NO and a molecular weight of 185.35. Pirfenidone is a white to pale yellow, non-hygroscopic powder and is more soluble in methanol, ethyl alcohol, acetone and chloroform than in water and 1.0 N HCl. Pirfenidone has a melting point of approximately 109° C. Reference herein to pirfenidone also includes a reference to a pharmaceutically acceptable salt of pirfenidone unless otherwise indicated or clear from context. In some embodiments, pirfenidone is in the form of pirfenidone hydrochloride. Pirfenidone and pharmaceutically acceptable salts thereof are commercially available (e.g., Genentech, Inc.).

“Ubenimex” refers to (2S)-2-[[(2S,3R)-3-amino-2-hydroxy-4-phenylbutanoyl]amino]-4-methylpentanoic acid, which is also known as N-[(2S,3R)-3-Amino-2-hydorxy-4-phenylbutyryl-L-leucine, the structure of which is shown below:

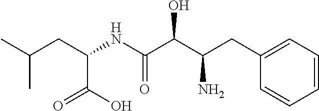 [SMILES: CC(C)C[C@H](NC(=O)[C@@H](O)[C@H](N)Cc1ccccc1)C(=O)O]

Ubenimex is a zwitterionic molecule that has a solubility of 1.27 mg/mL in water and has a melting point of approximately 251° C. Ubenimex is described in U.S. Pat. Nos. 4,029,547 and 4,052,449, incorporated by reference herein. Reference herein to ubenimex also includes a reference to a pharmaceutically acceptable salt of ubenimex unless otherwise indicated or clear from context. In some embodiments, ubenimex is in the form of ubenimex hydrochloride. Ubenimex and pharmaceutically acceptable salts thereof are commercially available (e.g., Tocris Bioscience). Ubenimex can also be prepared according to methods known in the art. See, e.g., U.S. Pat. No. 4,029,547, incorporated by reference herein. As used herein, ubenimex may be formulated as a solid pharmaceutical composition, or a liquid pharmaceutical composition, such as a composition comprising ubenimex in suspension, solution, or emulsion in an oily or aqueous vehicle, as described in PCT/US2017/054087, incorporated by reference herein.

All percentages are % w/w, unless otherwise specified. Unless otherwise indicated, “% weight” is percent weight of the specified component compared to total weight of the unit dosage (e.g., tablet or capsule). It will be appreciated that due to rounding or practical limits on quantitative measurements, reference to a quantity of API or excipient in a dosage form can include some variation, such as ±0.10% or ±0.5%.

### III. METHODS OF TREATING NAFLD AND NASH

In one aspect, the present disclosure provides a therapy for the treatment of NASH that comprises administering to a patient in need of treatment a therapeutically efficacious dose of pirfenidone. In some embodiments, the patient in need of treatment is a patient who has been diagnosed with NASH. In some embodiments, treatment with pirfenidone as described herein slows, stops, or reverses NASH disease progression.

In another aspect, the present disclosure provides a therapy for the prevention of NASH or for slowing the progression of NAFLD to NASH by administering to a patient in need of treatment a therapeutically efficacious dose of pirfenidone. In some embodiments, the patient in need of treatment is a patient who has been diagnosed with NAFLD.

### Patient Population

Patients likely to benefit from the therapies of the present disclosure can be readily identified by a variety of means discussed herein or known to those of skill in the art. In addition, methods for determining whether a patient is responding to this therapy are also provided. In some embodiments, abdominal imaging tests, including ultrasound examination, computerized tomography (CT), and/or magnetic resonance imaging (MRI) can be used to diagnose patients with the disease, e.g. evaluate whether the disease is present and its severity. Such a non-invasive diagnosis can be more definitively confirmed by liver biopsy, if desired. In some embodiments, one or more biomarkers is used to diagnose NAFLD or NASH. In some embodiments, a patient to be treated in accordance with the present disclosure has received a primary diagnosis of NASH or NAFLD and is not being treated with pirfenidone for any other condition for which it is currently indicated or in clinical development (e.g., certain cancer patients, PAH indication, or lymphedema indication).

NAFLD is characterized by significant lipid deposition in hepatocytes and is typically defined as either excessive fat accumulation in the liver (with more than 5% of hepatocytes containing visible intracellular triglycerides) or steatosis affecting at least 5% of the liver volume or weight. El-Kader et al., World J Hepatol, 2015, 7:846-858. In some embodiments, a patient to be treated has NAFLD. Some patients also exhibit abnormal liver function, e.g., as determined by the presence of elevated serum aspartate aminotransferase (ALT), gamma glutamyl transpeptidase, or alkaline phosphatase levels. In some embodiments, a patient to be treated has NAFLD and further has an elevated ALT level, an elevated gamma glutamyl transpeptidase, or an elevated alkaline phosphatase level (e.g., a level that is about 1.5- to 4-fold above the upper limit of normal). In some embodiments, a patient to be treated has NAFLD and has an ALT level, gamma glutamyl transpeptidase level, or alkaline phosphatase level that is within the upper limit of normal.

In some embodiments, NAFLD is diagnosed using an imaging test. In some embodiments, NAFLD is diagnosed using a scoring system such as but not limited to fatty liver index (in which a score >60 indicates a high risk for NAFLD), NAFLD liver fat score, NAFLD activity score, or hepatic steatosis index. In some embodiments, NAFLD is diagnosed using a NAFLD activity score (NAS), which provides a composite score based on the degree of steatosis (0-3), lobular inflammation (0-3), and hepatocyte ballooning (0-2). See, Kleiner et al., Hepatology, 2005, 41:1313-1321; Bugianesi et al., 3 Hepatology, 2016, 65:643-644.

NASH has been classified pathologically into type 1 and type 2 forms, of which the type 1 form is more commonly found in adult patients, while the type 2 form is more commonly found in children. Type 1 NASH is typically characterized by steatosis, hepatocyte ballooning, and perisinusoidal fibrosis. Type 2 NASH is typically characterized by steatosis, portal inflammation, and portal fibrosis. See, e.g., Schwimmer et al., Hepatology, 2005, 42:641-649. Further progression of NASH can lead to severe fibrosis, cirrhosis, and end-stage liver disease. In some embodiments, a patient to be treated has Type 1 NASH. In some embodiments, a patient to be treated has Type 2 NASH. In some embodiments, a patient to be treated has early-stage NASH. In some embodiments, a patient to be treated has middle-stage NASH. In some embodiments, a patient to be treated has late-stage NASH (e.g., has severe fibrosis and/or cirrhosis of the liver).

In some embodiments, NASH is diagnosed using an imaging test. In some embodiments, NASH is diagnosed using a scoring system such as but not limited to NAFLD activity score (e.g., a score of ≥5) or a steatosis, activity, and fibrosis (SAF) score, or a NAFLD fibrosis score; a serum biomarker (e.g., cytokeratin-18); or a combination thereof. See, Bedossa et al., Hepatology, 2012, 56:1751-1759; Arab et al., Gastroenterol Hepatol, 2017, 40:388-394. In some embodiments, fibrosis is detected and/or measured using elastography (e.g., Fibroscan®).

In some embodiments, a patient to be treated is identified by use of one or more biomarkers such as CK-18. CK-18 levels, whether measured by immunohistochemistry, histology from liver biopsies, or via measurement of plasma levels in patients or individuals suspected of being at risk for the disease, will typically be elevated, relative to the levels measured in healthy individuals, in subjects in need of treatment. While the invention is not to be limited to a particular or any proposed mechanism of action, decreased CK-18 levels in NASH patients would be expected to correlate with decreased liver cell apoptosis. Accordingly, patients with NAFLD or NASH who are treated with pirfenidone in accordance with the present disclosure should benefit from decreased liver cell apoptosis, relative to receiving no treatment or standard of care.

In some embodiments, a patient to be treated is a human adult. In some embodiments, a patient to be treated is a human child under 18 years of age (e.g., from age 2 to age 17).

In some embodiments, a patient to be treated does not have cancer. In some embodiments, a patient to be treated does not have acute non-lymphocytic leukemia. In some embodiments, a patient to be treated does not have lymphedema. In some embodiments, a patient to be treated does not have pulmonary arterial hypertension.

### Dosage Regimen

In some embodiments, pirfenidone is administered at a total daily dose that is about 3600 mg or less (e.g., less than 3500 mg, less than 3400 mg, less than 3300 mg, less than 3200 mg, less than 3100 mg, less than 3000 mg, less than 2900 mg, less than 2800 mg, less than 2700 mg, less than 2600 mg, less than 2500 mg, less than 2400 mg, less than 2300 mg, less than 2200 mg, less than 2100 mg, less than 2000 mg, less than 1900 mg, less than 1800 mg, less than 1700 mg, less than 1600, less than 1500 mg, less than 1400 mg, less than 1300 mg, less than 1200 mg, less than 1100 mg, less than 1000 mg, less than 900 mg, or less than 800 mg). As used herein, the dose amount refers to the amount of active ingredient administered per dose, not the amount of the pharmaceutical formulation. In some embodiments, pirfenidone is administered at a total daily dose in the range of about 267 mg to about 2403 mg, e.g., from about 100 mg to about 2403 mg, from about 200 mg to about 2403 mg, from about 300 mg to about 2403 mg, from about 400 mg to about 2403 mg, from about 500 mg to about 2403 mg, from about 600 mg to about 2403 mg, from about 700 mg to about 2403 mg, from about 800 mg to about 2403 mg, from about 900 mg to about 2403 mg, from about 1000 mg to about 2403 mg, from about 1100 mg to about 2403 mg, or from about 1197 mg to about 2403 mg. This daily dose may be administered all at once: one time daily, although in some embodiments the once daily dose (QD administration) will be at least 801 mg or more. For BID administration, in some embodiments the daily dose will be 50-1800 mg, e.g. about 400 mg to about 1800 mg two times daily, although in some embodiments the dose will be at least 598 mg or more for BID administration. For TID administration, in some embodiments the daily dose will be 30-800 mg, e.g. about 200 mg to about 800 mg three times daily, although in some embodiments the dose will be at least 800 mg or more for TID administration. In some embodiments, pirfenidone is administered at a daily dose of at least 801 mg, at least 1197 mg, at least 1602 mg, at least 2403 mg, at least 3600 mg, or at least 4005 mg. In some embodiments, pirfenidone is administered at a daily dose of about 100 mg, 200 mg, 300 mg, 400 mg, 500 mg, 600 mg, 700 mg, 800 mg, 801 mg, 900 mg, 1000 mg, 1100 mg, 1197 mg, 1200 mg, 1300 mg, 1400 mg, 1500 mg, 1600 mg, 1602 mg, 1700 mg, 1800 mg, 1900 mg, 2000 mg, 2100 mg, 2200 mg, 2300 mg, 2400 mg, 2403 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, or greater than 5000 mg.

In some embodiments, pirfenidone is administered at a dosage of about 801 mg to 2403 mg QD, e.g., about 800 mg to 2400 mg QD, about 900 mg to about 2400 mg QD, about 1000 mg to about 2400 mg QD, about 1100 mg to about 2400 mg QD, about 1197 mg to about 2400 mg QD, about 1200 mg to about 2400 mg QD, about 1300 mg to about 2400 mg QD, about 1400 mg to about 2400 mg QD, about 1500 mg to about 2400 mg QD, about 1600 mg to about 2400 mg QD, about 1700 mg to about 2400 mg QD, about 1800 mg to about 2400 mg QD, about 1900 mg to about 2400 mg QD, about 2000 mg to about 2400 mg QD, about 2100 mg to about 2400 mg QD, about 2200 mg to about 2400 mg QD, or about 2300 mg to about 2400 mg QD. In some embodiments, pirfenidone is administered at dosage of about 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 267 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 801 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1197 mg, 1200 mg, 1210 mg, 1220 mg, 1230 mg, 1240 mg, 1250 mg, 1260 mg, 1270 mg, 1280 mg, 1290 mg, 1300 mg, 1310 mg, 1320 mg, 1330 mg, 1340 mg, 1350 mg, 1360 mg, 1370 mg, 1380 mg, 1390 mg, 1400 mg, 1410 mg, 1420 mg, 1430 mg, 1440 mg, 1450 mg, 1460 mg, 1470 mg, 1480 mg, 1490 mg, 1500 mg, 1510 mg, 1520 mg, 1530 mg, 1540 mg, 1550 mg, 1560 mg, 1570 mg, 1580 mg, 1590 mg, 1600 mg, 1602 mg, 1610 mg, 1620 mg, 1630 mg, 1640 mg, 1650 mg, 1660 mg, 1670 mg, 1680 mg, 1690 mg, 1700 mg, 1710 mg, 1720 mg, 1730 mg, 1740 mg, 1750 mg, 1760 mg, 1770 mg, 1780 mg, 1790 mg, 1800 mg, 1810 mg, 1820 mg, 1830 mg, 1840 mg, 1850 mg, 1860 mg, 1870 mg, 1880 mg, 1890 mg, 1900 mg, 1910 mg, 1920 mg, 1930 mg, 1940 mg, 1950 mg, 1960 mg, 1970 mg, 1980 mg, 1990 mg, 2000 mg, 2010 mg, 2020 mg, 2030 mg, 2040 mg, 2050 mg, 2060 mg, 2070 mg, 2080 mg, 2090 mg, 2100 mg, 2110 mg, 2120 mg, 2130 mg, 2140 mg, 2150 mg, 2160 mg, 2170 mg, 2180 mg, 2190 mg, 2200 mg, 2210 mg, 2220 mg, 2230 mg, 2240 mg, 2250 mg, 2260 mg, 2270 mg, 2280 mg, 2290 mg, 2300 mg, 2310 mg, 2320 mg, 2330 mg, 2340 mg, 2350 mg, 2360 mg, 2370 mg, 2380 mg, 2390 mg, 2400 mg, 2403 mg, or 2410 mg QD.

In some embodiments, pirfenidone is administered at a dosage of about 400.5 mg to 1201.5 mg BID, e.g., about 400 mg to about 1200 mg BID, about 500 mg to about 1200 mg BID, about 600 mg to about 1200 mg BID, about 700 mg to about 1200 mg BID, about 800 mg to about 1200 mg BID, about 900 mg to about 1200 mg BID, about 1000 mg to about 1200 mg BID, or about 1300 mg to about 1200 mg BID. In some embodiments, pirfenidone is administered at dosage of about 400 mg, 400.5 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 598.5 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 801 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1200 mg, or 1210 mg BID.

In some embodiments, pirfenidone is administered at a dosage of about 267 mg to 801 mg TID, e.g., about 200 mg to about 800 mg, about 267 mg to about 800 mg TID, about 300 mg to about 800 mg TID, about 400 mg to about 800 mg TID, about 500 mg to about 800 mg TID, about 600 mg to about 800 mg TID, or about 700 mg to about 800 mg TID. In some embodiments, pirfenidone is administered at dosage of about 200 mg, 210 mg, 220 mg, 230 mg, 240 mg, 250 mg, 260 mg, 267 mg, 270 mg, 280 mg, 290 mg, 300 mg, 310 mg, 320 mg, 330 mg, 340 mg, 350 mg, 360 mg, 370 mg, 380 mg, 390 mg, 399 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 801 mg, 810 mg TID.

Formulations and unit dosage forms of pirfenidone are further disclosed in Section IV below. In some embodiments, pirfenidone is administered orally. In some embodiments, pirfenidone is administered orally in a therapeutically effective dose at least once and no more than thrice daily on consecutive days for at least a week and typically longer. In some embodiments, pirfenidone is administered alone (i.e., not in combination with another medication) for part or all of the treatment period. In some embodiments, pirfenidone is administered in combination with ubenimex

In some embodiments, pirfenidone is administered as a pharmaceutical formulation suitable for oral administration. In some embodiments, pirfenidone is administered as a pharmaceutical formulation suitable for parenteral administration. In some embodiments, pharmaceutical formulations for use according to the present disclosure are prepared for oral administration and in an immediate release form suitable for QD, BID, or TID administration, and the dosage regimen is selected within the ranges provided herein in accordance with a variety of factors including type, species, age, weight, sex and medical condition of the patient; the severity of the condition to be treated; the route of administration; the renal and hepatic function of the patient; and the particular formulation employed. An ordinarily skilled physician or veterinarian can readily determine and prescribe the effective amount of the drug required to prevent, counter or arrest the progress of the condition in view of the teachings herein.

### Duration of Treatment, Treatment Endpoints, and Monitoring Efficacy

In some embodiments, treatment with pirfenidone (and optionally a second therapeutic agent) is administered for a predetermined time, an indefinite time, or until an endpoint is reached. In some embodiments, treatment is for at least 15 days, at least 60 days or two months, at least 90 days or three months, at least 120 days or four months, at least 150 days or five months, at least 180 days or six months, at least 52 weeks or 1 year, at least 72 weeks, or for the remainder of the subject's life. In some embodiments, treatment is continued for at least one year. In some embodiments, pirfenidone is administered (e.g., by consecutive daily administration of pirfenidone as described herein) for at least 2 weeks, for at least a month, for at least 3 months, or for at least 6 months to at least a year. In other embodiments, treatment is continued for the rest of the subject's life or until administration is no longer effective in providing a meaningful therapeutic benefit. In some embodiments, treatment is administered on a continuous daily basis. In some embodiments, treatment is administered on a near continuous daily basis (e.g., pirfenidone treatment is administered to a patient daily but the patient may occasionally miss a day of treatment).

In some embodiments, a patient undergoes an initial 2-week titration regimen, wherein for treatment days 1 through 7 pirfenidone is administered at a total daily dose of 801 mg, for treatment days 8 through 14 pirfenidone is administered at a total daily dose of 1602 mg, and for treatment days 15 and onward pirfenidone is administered at a total daily dose of 2403 mg. In one embodiment, the total daily dose for treatment days 1 through 7 is administered at a dosage of 267 mg TID, the total daily dose for treatment days 8-14 is administered at a dosage of 534 mg TID, and the total daily dose for treatment days 15 and onward is administered at a dosage of 801 mg TID.

In some embodiments, generally continuous (or near continuous) daily dosing is continued until treatment appears to no longer have a beneficial effect or until unacceptable side effects appear. Many patients will take the medication for at least 2 weeks, at least a month, and at least a year or longer. In some instances, a patient will take the medication from approximately 6 months to approximately 1 year. In some instances, a patient will take the medication for greater than 1 year. Many patients will take the medication for the rest of their lives.

In some embodiments, treatment according to the methods described herein results in an improvement in one or more parameters such as, but not limited to, an improvement in NAS (ballooning and inflammation) and/or fibrosis; an improvement in SAF (steatosis, activity, and fibrosis) score; complete resolution of steatohepatitis; no worsening of fibrosis; an improvement in fibrosis without a worsening of steatohepatitis; or an increased time to disease progression as measured by histopathologic assessment of progression to cirrhosis, death, liver transplant, hepatocellular carcinoma, and decompensation events such as hepatic encephalopathy, variceal bleeding requiring hospitalization, ascites requiring intervention, and spontaneous bacteria peritonitis. In some embodiments, treatment according to the methods described herein results in an improvement (i.e., a reduction) in hepatocyte ballooning. In some embodiments, hepatocyte ballooning is visualized using hematoxylin and eosin straining.

In some embodiments, treatment according to the methods described herein results in an improvement in one or more biomarkers of NAFLD or NASH, such as but not limited to markers of apoptosis (e.g., CK-18 fragments), adipokines (e.g., adiponectin, leptin, resistin, or visfatin), inflammatory markers (e.g., TNF-α, IL-6, chemo-attractant protein-1, or high sensitivity C-reactive protein). See, e.g., Neuman et al., Can J Gastroenterol Hepatol, 2014, 28:607-618; Castera et al., Nat Rev Gastroenterol Hepatol., 2013, 10:666-675. In some embodiments, biomarker values are measured using a sample that comprises a fluid, e.g., blood, plasma, serum, urine, or cerebrospinal fluid. In some embodiments, biomarker values are measured using a sample that comprises cells and/or tissues, e.g., hepatocytes or liver tissue. In some embodiments, treatment results in an improvement in the biomarker CK-18. In some embodiments, treatment results in a reduction in plasma CK-18 levels in the subject.

In some embodiments, a patient is monitored during the course of pirfenidone therapy using a diagnostic test as described herein (e.g., using abdominal imaging tests). In some embodiments, the method further comprises continuing a course of therapy (e.g., a dosage of pirfenidone as described herein). In some embodiments, the method further comprises tapering, reducing, or stopping the administered amount of pirfenidone if the diagnosis warrants, e.g. when a cure is effected, a lower dose appears to be safer or equally efficacious as a higher dose, or no continuing therapeutic effect is expected. In some embodiments, the methods can comprise increasing the administered amount of pirfenidone if it is determined not to be efficacious, as well as stopping therapy if it is determined dose escalation or continued dosing at any dose is unlikely to be efficacious.

In some embodiments where the patient is undergoing treatment in accordance with the present disclosure, indications of NASH by abdominal imaging, ultrasound examination, magnetic resonance imaging, CT scan, and/or biopsy may be less than those measured in the patient prior to treatment, which is indicative that the patient is responding positively to the therapy. In cases where the patient is responding positively to a therapy of the present disclosure, the therapy is continued until the presence of the condition is reduced to a level comparable to a normal control level. Optionally, the therapy is continued to maintain alleviation of NASH symptoms. Alternatively, the therapy is continued until a desired level of steatosis is achieved in the patient (including the absence of steatosis). Treatment may be continued for so long as it is determined to be efficacious using assessment by abdominal imaging, ultrasound examination, magnetic resonance imaging, CT scan, and/or biopsy. The treatment may be determined to be efficacious through measured improvement in one or more of steatosis, ballooning, and necroinflammation. In one embodiment, the treatment is determined to be efficacious through measured improvement indicated by induced reduction in ballooning. In one embodiment, the treatment is determined to be efficacious through measured improvement indicated by a reduction in inflammation. In one embodiment, the treatment is determined to be efficacious through measured improvement indicated by at least one of reduced serum ALT levels, improved insulin sensitivity, reduced steatosis, reduced inflammation, and reduced fibrosis. In one embodiment, the treatment is determined to be efficacious through measured improvement indicated by induced regression or reversal of fibrosis and/or cirrhosis.

In some embodiments, treatment results in an improvement in one or more parameters (e.g., a reduction in NAS or SAF score, a reduction in hepatocyte ballooning, a reduction in fibrosis, or a reduction in CK-18 levels) of at least 10%, at least 20%, at least 30%, at least 40%, at least 50%, at least 60%, at least 70%, at least 80%, or at least 90% as compared to a control value. In some embodiments, treatment results in an improvement in one or more parameters of at least 2-fold, at least 3-fold, at least 4-fold, at least 5-fold, at least 6-fold, at least 7-fold, at least 8-fold, at least 9-fold, or at least 10-fold as compared to a control value. In some embodiments, the control value is a baseline value for the subject that is determined prior to the onset of treatment.

In some embodiments, the present disclosure provides methods of determining efficacy of a NASH treatment in a subject in need thereof by (a) measuring the level and severity of NASH via abdominal imaging, ultrasound examination, magnetic resonance imaging, CT scan, and/or liver biopsy in a subject in need thereof, where the level and severity of NASH is measured after treatment has started, (b) comparing the level and severity of NASH as measured in step (a) to a baseline level and severity of NASH, where the baseline level and severity is measured in the same subject before treatment is begun, and (c) determining the efficacy of the NASH treatment based on the comparison step.

Furthermore, in some embodiments the present disclosure provides methods of determining efficacy of a NASH treatment in a subject in need thereof by (a) measuring the level and severity of NASH in a subject in need thereof after treatment has begun, (b) comparing the level and severity of the NASH to a reference value, where the reference value represents an average value determined from a population of patients suffering from NASH, and (c) determining the efficacy of the NASH treatment based on the comparison step. In some embodiments, efficacy of therapy is determined by liver biopsy and analysis to evaluate NAFLD Activity Score (NAS) and fibrosis; the transjugular liver biopsy method can be employed for this purpose. Suitable patients include patients with biopsy proven NASH, patients at high risk for NASH, patients with a NAS greater than or equal to 4, NASH patients with liver fibrosis, and NASH patients with liver fibrosis of stage 2 or greater.

In some embodiments, patients responding to therapy in accordance with the invention are expected to show at least a slowing of any increase in CK-18 levels as therapy continues. In some embodiments, those patients responding most favorably to therapy will have CK-18 levels that stabilize and decline over time as full therapeutic benefit is realized. Thus, in some embodiments, the present disclosure provides methods of determining efficacy of a NASH treatment in a subject in need thereof by (a) measuring the level and severity of NASH via measuring the level of the biomarker CK-18 in a sample from the sample from the subject (e.g., a blood, plasma, or tissue sample), wherein the level and severity of NASH is measured after treatment has started, (b) comparing the level and severity of NASH measured in (a) to a baseline level and severity of NASH in the subject that is measured in the same subject before treatment is begun, and (c) determining the efficacy of the NASH treatment based on the comparison step; wherein a plateau or decrease in CK-18 levels is indicative of efficacy of the NASH treatment.

### IV. COMPOSITIONS, UNIT DOSAGE FORMS, AND KITS COMPRISING PIRFENIDONE

In another aspect, compositions, unit dosage forms, pharmaceutical packages, and kits comprising pirfenidone for use in the methods described herein are provided. In some embodiments, the formulations, unit dosage forms, pharmaceutical packages, and/or kits are for use in treating NASH. In some embodiments, the formulations, unit dosage forms, pharmaceutical packages, and/or kits are for use in delaying or preventing the progression of NAFLD to NASH in a subject having NAFLD.

In some embodiments, the compositions, unit dosage forms, pharmaceutical packages, and/or kits comprise pirfenidone or a pharmaceutically acceptable salt thereof. In some embodiments, the composition, unit dosage form, pharmaceutical package, or kit comprises pirfenidone or pirfenidone hydrochloride. In some embodiments, a composition or unit dosage form comprises pirfenidone or a pharmaceutically acceptable salt thereof in an amount from about 10% to 50%, or from about 20% to about 40%, or about 30% to about 35%, or from about 15% to about 25% by weight of the total composition. In some embodiments, pirfenidone is provided as a liquid liposomal formulation suitable for parenteral administration. In some embodiments, a composition or unit dosage form comprises pirfenidone or a pharmaceutically acceptable salt thereof in a concentration from about 50-3600 mg/ml, 100-3200 mg/ml, 200-2403 mg/ml, 267-2200 mg/ml, 300-1800 mg/ml, 399-1201.5 mg/ml, or 534-801 mg/ml. In some embodiments, pirfenidone is formulated in a pharmaceutical composition comprising one or more pharmaceutically acceptable excipients. In some embodiments, an excipient comprises a solubilizing agent, a stabilizing agent, a diluent, an inert carrier, a preservative, a binder, a disintegrant, a coating agent, a flavoring agent, or a coloring agent. Suitable pharmaceutical compositions, formulations, and unit dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). Typically, pharmaceutical formulations of the disclosure comprise pirfenidone and one or more pharmaceutically acceptable (approved by a state or federal regulatory agency for use in humans, or is listed in the U.S. Pharmacopia, the European Pharmacopia) excipients or carriers.

In some embodiments, at least one excipient is chosen to provide one or more beneficial physical properties to the formulation, such as increased stability and/or solubility of the active agent(s). Examples of suitable excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as TWEEN®, PLURONIC®, or polyethylene glycol (PEG); carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

In some embodiments, pirfenidone is formulated for parenteral administration in a liposome for targeting to the lung (see Meng H, Xu Y. Pirfenidone-loaded liposomes for lung targeting: preparation and in vitro/in vivo evaluation. Drug Des Devel Ther. 2015;9:3369-3376). In some embodiments, the pirfenidone liposome formulation further comprises one or more formulatory agents such as suspending, stabilizing, and/or dispersing agents. In some embodiments, solutions or suspensions comprising liposomes used for the delivery of pirfenidone can include the following components: a sterile diluent such as water for injection, saline solution, fixed oils, polyethylene glycols, glycerine, propylene glycol, polysorbate, tocopherol polyethylene glycol succinate (TPGS), or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. These preparations can be enclosed in ampoules, disposable syringes or multiple dose vials made of glass or plastic.

In some embodiments, pirfenidone is formulated for immediate release. As used herein, “immediate release” means a drug formulation that provides for the release of at least a majority of the drug within a relatively short period of time after administration (e.g., within about one hour). In some embodiments, an immediate release formulation provides for the release of at least about 80% of the drug within about 30-60 minutes after administration.

In some embodiments, pirfenidone is formulated for sustained release or extended release. As used herein, “sustained release” and “extended release” means a drug formulation that provides for gradual release of a drug over an extended period of time, and typically, although not necessarily, results in substantially constant blood levels of a drug over an extended time period. In some embodiments, a sustained release formulation provides for a substantially constant blood level of the agent over a time period in the range of about 4 to about 12 hours, typically in the range of about 6 to about 10 hours. For example, a sustained release formulation can provide a very gradual increase in blood level of a drug following administration such that peak blood level is not reached until at least 4-6 hours have elapsed, with the rate of increase of blood level drug approximately linear, followed by a sustained period of peak blood levels and then by an equally gradual decrease in blood levels at the end of the sustained release period.

In some embodiments, pirfenidone is formulated for delayed release. As used here, “delayed release” refers to a drug formulation that, following administration to a patient, provides a measurable time delay before drug is released from the formulation into the subject's body.

In some embodiments, oral pirfenidone formulations are formulated as immediate release preparations, and are conveniently packaged, for example, in unit dosage forms in the form of a pill, capsule, or tablet, which in turn may be in a pill bottle or blister packaging. Dosages and desired drug concentration of pharmaceutical compositions of the disclosure may vary depending on the particular use envisioned. The determination of the appropriate dosage or route of administration is well within the skill of one in the art. Suitable dosages are also described in Section III above. In some embodiments, pirfenidone is provided in oral form in a fixed dosage amount in a unit dosage form that is a pill, tablet, or capsule containing amounts of pirfenidone ranging from 100-2500 mg, 200-2403 mg, 267-2400 mg, 300-2300 mg, 400-2200 mg, 500-2100 mg, 534-2000 mg, 600-1900 mg, 700-1800 mg, 800-1700 mg, 900-1600 mg, 1000-1500 mg, 1100-1400 mg, or 1200-1300 mg. The particular unit dosage form will depend on the dose to be administered, which may depend on whether the patient is an adult or child or upon the severity of the disease.

In some embodiments, pirfenidone liposome formulations are formulated as sustained release preparations, and are conveniently packaged, for example, in unit dosage forms in form of a vial, ampoule, syringe, bottle or other liquid compatible containers. In some embodiments, pirfenidone liposome formulations are provided in pirfenidone concentrations ranging from 50-3600 mg/ml, 100-3200 mg/ml, 200-2403 mg/ml, 267-2200 mg/ml, 300-1800 mg/ml, 339-1201.5 mg/ml, or 534-801 mg/ml. The particular unit dosage form will depend upon the dose to be administered, which may depend on whether the patient is an adult or child or upon the severity of the disease.

In some embodiments, most adult patients (60-100 kg or more) will receive therapeutic benefit from receiving a pirfenidone dose of 801 mg three times daily. In some embodiments, most adult patients will receive therapeutic benefit from a single dose in the range of 801-2403 mg per day, with some achieving full therapeutic effect with 801-2403 mg at least once daily, 400.5-1201.5 mg twice daily, or 267-801 mg three times daily. However, minimal doses of 200 mg, 339 mg, 534 mg, 598.5 mg, 801 mg, 1200 mg, and 1800 mg administered on these schedules (QD, BID, and TID) may be approved for clinical use. Some patients will administer the prescribed dose with each meal. Some patients will administer this dose before meals. Some patients will administer this dose as a chronic medication; it is anticipated that some patients will take the drug every day for periods of 6 months or longer.

The present disclosure also provides a variety of specific unit dosage forms suitable for use in the treatment methods described herein. For example, pirfenidone can be administered in unit dosage forms containing from 100, 200, 267, 300, 399, 400, 500, 534, 598.5, 600, 700, 800, 801, 900, 1000, 1100, 1200, 1201.5, 1300, 1400, 1500, 1600, 1700, 1800, 1900, 2000, 2100, 2200, 2300, 2400, 2403, or 2500 mg of pirfenidone, which are suitable for delivery one, twice, or three times a day to provide the daily dose prescribed by the physician in accordance with this disclosure. In a preferred embodiment, pirfenidone is administered in a unit dosage form of 801 mg TID. Moreover, previous formulations and unit dosage forms of pirfenidone can be used in the methods of the disclosure. Pirfenidone has been marketed for the treatment of idiopathic pulmonary fibrosis under the brand name Esbriet®. In various embodiments of the disclosure, pirfenidone, including but not limited to pirfenidone in the Esbriet® 267 mg, 534 mg, or 801 mg unit dosage forms commercially available, is administered to a patient with NASH.

In some embodiments, pharmaceutical packages and kits comprising pirfenidone and a second therapeutic agent comprising ubenimex are provided. In some embodiments, the pharmaceutical package or kit comprises immediate release unit dosage forms of pirfenidone and ubenimex. In some embodiments, the pharmaceutical package or kit comprises extended release unit dosage forms of pirfenidone and ubenimex. In some embodiments, the pharmaceutical package or kit comprises an extended release unit dosage form of pirfenidone and immediate release unit dosage forms of ubenimex.

In some embodiments, the pharmaceutical package or kit is for use in treating NASH. In some embodiments, the pharmaceutical package or kit is for use in delaying or preventing the progression of NAFLD to NASH in a subject having NAFLD. In some embodiments, the pharmaceutical package or kit further comprises instructional materials for use according to a method disclosed herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to interne sites that provide such instructional materials.

### V. COMBINATION THERAPIES FOR THE TREATMENT AND/OR THE PREVENTION OF NASH

In another aspect, pirfenidone is administered in combination with an additional therapeutic agent. Combination therapies for the treatment and/or the prevention of NASH may further include administration of pirfenidone in combination with one or more lifestyle changes, such as exercise to reduce body weight and/or activities to improve physical and/or mental health.

In some embodiments, pirfenidone is administered in combination with ubenimex or a pharmaceutically acceptable salt thereof. In some embodiments, pirfenidone is administered a dosage of about 267 mg to about 2403 mg QD (e.g., QD, BID or TID) and ubenimex is administered at a dosage of about 10 to 500 mg (e.g., QD or BID). In a preferred embodiment, pirfenidone is administered at a daily dosage of 2403 mg, and ubenimex is administered at a dosage of 450 mg. In another preferred embodiment, pirfenidone is administered as an immediate release formulation at a dosage of 801 mg TID, and ubenimex is administered as an immediate release formulation at a dosage of 150 mg TID. In a further preferred embodiment, pirfenidone is administered as an extended release formulation at a dosage of 2403 mg QD, and ubenimex is administered as an extended release formulation at a dosage of 450 mg QD. In one embodiment, at least one of pirfenidone and ubenimex is administered as an extended release formulation.

The beneficial effect of the combination or pirfenidone and ubenimex may include, but is not limited to, pharmacokinetic or pharmacodynamic co-action resulting from the combination of therapeutic agents. Such combination therapies can result in an improved parameter such as, but not limited to, improved NASH scores (e.g., as measured by the NAFLD Activity Score), decreased fibrosis scores, and decreased serum alanine aminotransferase (ALT) levels; biochemical and histological improvements in NASH; and improvements in steatosis, inflammation, and fibrosis.

Administration of pirfenidone in combination with ubenimex typically is carried out over a defined time period (e.g., over a period of days, weeks, months, or years depending upon the combination selected). Combination therapies of the present invention includes administration the therapeutic agents in a sequential manner, wherein each therapeutic agent is administered at a different time, as well as administration of the two therapeutic agents in a substantially simultaneous manner. Substantially simultaneous administration can be accomplished, for example, by administering to the subject a single capsule having a fixed ratio of each therapeutic agent, or in separate capsules for each of the therapeutic agents. In some embodiments, the pirfenidone and the ubenimex are formulated separately. In some embodiments, the pirfenidone and the ubenimex are formulated in a single composition.

Sequential or substantially simultaneous administration of each therapeutic agent can be effected by any appropriate route, including, but not limited to, oral routes and parenteral routes. The two therapeutic agents can be administered by the same route or by different routes. For example, pirfenidone may be administered parenterally while ubenimex may be administered orally. Alternatively, for example, all therapeutic agents may be administered orally or all therapeutic agents may be administered parenterally. For the combination therapies disclosed herein, it is contemplated that each agent can be administered in an “immediate release” manner or in a “controlled release” or “delayed release” manner.

As a non-limiting example, a combination dosage form for once-daily administration may contain in the range of 801 mg to 2403 mg of pirfenidone in a controlled release (e.g., sustained or extended release) form, and in the range of 150 mg to 450 mg of ubenimex in a controlled release (e.g., sustained or extended release), wherein each active agent is present in a suitable weight ratio. For example, in one embodiment a suitable weight ratio of ubenimex to pirfenidone is about 1:1, about 1:2, about 1:3, about 1:4, about 1:8, about 1:10, or about 1:16.

Combination therapy also includes the administration of pirfenidone and ubenimex as described above in further combination with other non-drug therapies (e.g., surgery or physical therapy). Where a combination therapy comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### EXAMPLES

The following examples are offered to illustrate, but not to limit, the claimed invention.

### Example 1: Pharmaceutical Composition for the Treatment and/or Prevention of Non-Alcoholic Steatoshepatitis (NASH)

A pharmaceutical composition for the treatment and/or the prevention of NASH is provided comprising pirfenidone (5-methyl-1-phenyl-2-1-(H)-pyridone; also known as 5-methyl-1-phenyl-2-(1H)-pyridone), or a pharmaceutically acceptable salt thereof, including but not limited to pirfenidone hydrochloride.

### Example 2: Pharmaceutical Composition for the Treatment and/or Prevention of NASH

A pharmaceutical composition for the treatment and/or the prevention of NASH is provided comprising pirfenidone, or a pharmaceutically acceptable salt thereof, and ubenimex ((2S)-2-[[(2S,3R)-3-amino-2-hydroxy-4-phenylbutanoyl]amino]-4-methylpentanoic acid; also known as N-[(2S,3R)-3-Amino-2-hydorxy-4-phenylbutyryl-L-leucine), or a pharmaceutically acceptable salt thereof.

### Example 3: Method and Therapy for the Treatment of NASH

A NASH patient is administered pirfenidone in a maximum daily dose of up to 3600 mg, and preferably administered pirfenidone in a dose of 801 mg TID, for not less than 15 days, and preferably for at least 30 day, at least 60 days, at least 90 days, at least 6 months, at least 1 year, or at least greater than one year, including the remainder of the patient's life. In some cases, the NASH patient is administered an oral formulation of pirfenidone. In some cases, the oral formulation is an immediate-release formulation. In some cases, the oral formulation is a controlled-release formulation (e.g., delayed or extended release). In some cases, the NASH patient is administered a liquid formulation of pirfenidone for parenteral administration. In some cases, the liquid formulation is a controlled-release formulation. In some instances, the liquid formulation comprises a liposomal formulation of pirfenidone.

### Example 4: Method and Therapy for the Prevention of NASH

A FLD or NAFDL patient is administered pirfenidone in a maximum daily dose of up to 3600 mg, and preferably administered pirfenidone in a dose of 801 mg TID, for not less than 15 days, and preferably for at least 30 day, at least 60 days, at least 90 days, at least 6 months, at least 1 year, or at least greater than one year, including the remainder of the patient's life. In some cases, the FLD or NAFLD patient is administered an oral formulation of pirfenidone. In some cases, the oral formulation is an immediate-release formulation. In some cases, the oral formulation is a controlled-release formulation (e.g., delayed or extended release). In some cases, the FLD or NAFLD patient is administered a liquid formulation of pirfenidone for parenteral administration. In some cases, the liquid formulation is a controlled-release formulation. In some cases, the liquid formulation comprises a liposomal formulation of pirfenidone.

### Example 5: Method and Therapy for the Treatment and/or Prevention of NASH

A NASH, FLD, or NAFLD patient is administered a therapeutically effective amount of pirfenidone in combination with a therapeutically effective amount of ubenimex as part of a combination therapy. In some cases, the patient is administered pirfenidone in a maximum daily dose of up to 3600 mg, and preferably administered pirfenidone in a dose of 801 mg TID, and further administered ubenimex in a maximum daily dose of up to 450 mg, and preferably administered ubenimex in a dose of 150 mg TID. In some cases, pirfenidone and ubenimex are concomitantly administered to the patient in immediate-release formulations. In some cases, pirfenidone and ubenimex are administered as controlled-release (e.g., delayed or extended release) formulations. In some cases, pirfenidone and ubenimex are administered as oral formulations. In some cases, at least one of pirfenidone and ubenimex is administered as a liquid formulation for parenteral administration. For example, in one case pirfenidone is administered in a liposome. In one case, pirfenidone and ubenimex are both administered in a liquid formulation. In one case, pirfenidone and ubenimex are both administered in one or more liposomal formulations.

The patient is generally treated with the combination therapy for not less than 15 days, and preferably for at least 30 day, at least 60 days, at least 90 days, at least 6 months, at least 1 year, or at least greater than one year, including the remainder of the patient's life.

While this disclosure has been particularly shown and described with references to preferred embodiments thereof, it will be understood by those skilled in the art that various changes in form and details may be made therein without departing from the scope of the disclosure encompassed by the appended claims.

All publications, patents, patent applications, or other documents cited herein are hereby incorporated by reference in their entirety for all purposes to the same extent as if each individual publication, patent, patent application, or other document was individually indicated to be incorporated by reference for all purposes.

What is claimed is:

1. A method of treating non-alcoholic steatohepatitis (NASH), the method comprising administering to a subject in need of treatment a therapeutically effective amount of pirfenidone.

2. The method of claim 1, wherein the subject has early-stage or middle-stage NASH.

3. The method of claim 1, wherein the pirfenidone is administered at a total daily dose in the range of 100 mg to 3600 mg.

4. The method of claim 1, wherein the pirfenidone is administered at a daily dose of about 300 mg to about 2403 mg.

5. The method of claim 4, wherein the pirfenidone is administered at dose of about 801 mg to about 2403 mg QD.

6.-7. (canceled)

8. The method of claim 1, wherein the pirfenidone is administered for at least 15 weeks.

9. (canceled)

10. The method of claim 1, wherein treatment results in a reduction in plasma CK-18 levels in the subject.

11. The method of claim 1, wherein treatment results in a reduction in hepatocyte ballooning in the subject.

12. The method of claim 1, wherein the pirfenidone is administered in combination with ubenimex.

13. The method of claim 12, wherein pirfenidone is administered at a dose of 801 mg TID, and ubenimex is administered at a dose of 150 mg TID.

14.-19. (canceled)

20. A method of delaying or preventing the progression of non-alcoholic fatty liver disease (NAFLD) to NASH in a subject having NAFLD, the method comprising administering to the subject a therapeutically effective amount of pirfenidone.

21. The method of claim 20, wherein treatment results in a reduction in hepatocyte ballooning in the subject.

22. (canceled)

23. The method of claim 20, wherein the pirfenidone is administered at a daily dose of about 100 mg to about 2403 mg.

24. The method of claim 23, wherein the pirfenidone is administered at a dose of about 801 mg to about 2403 mg QD.

25. The method of claim 23, wherein the pirfenidone is administered at a dose of about 400.5 mg to about 1201.5 mg BID.

26. The method of claim 23, wherein the pirfenidone is administered at a dose of about 267 mg to about 801 mg TID.

27. The method of claim 20 wherein the pirfenidone is administered for at least 15 weeks.

28. (canceled)

29. A method of decreasing hepatocyte ballooning in a subject having NASH, the method comprising administering to the subject a therapeutically effective amount of pirfenidone for at least 15 weeks.

30. The method of claim 29, wherein the pirfenidone is administered for at least 52 weeks.

31. A method of decreasing inflammation and/or fibrosis in a subject having NASH, the method comprising administering to the subject a therapeutically effective amount of pirfenidone for at least 24 weeks.

32. (canceled)

33. The method of claim 31, wherein the pirfenidone is administered in combination with ubenimex.

34. A method of treating NASH or slowing the progression of NAFLD to NASH, the method comprising administering a combination therapeutic composition comprising pirfenidone and ubenimex.

35. The method of claim 34, wherein the combination therapeutic composition is administered for at least 15 weeks.

36.-37. (canceled)

38. A pharmaceutical package comprising unit dosage forms of pirfenidone and further comprising unit dosage forms of ubenimex.

39. The pharmaceutical package of claim 38, wherein each unit dosage form of pirfenidone comprises pirfenidone in an amount from about 267 mg to about 801 mg, and wherein each unit dosage form of ubenimex comprises ubenimex in an amount from about 75 mg to about 150 mg.

40. The pharmaceutical package of claim 38, wherein at least one of the pirfenidone and the ubenimex is formulated for immediate release.

41. The pharmaceutical package of claim 38, wherein at least one of the pirfenidone and the ubenimex is formulated for controlled release.

42. The pharmaceutical package of claim 38, wherein at least one of the pirfenidone and the ubenimex is a solid formulation in the form of a tablet, a capsule, or a pill.

43. The pharmaceutical package of claim, wherein at least one of the pirfenidone and the ubenimex is a liquid formulation for oral or parenteral administration.

44. (canceled)
